(19) United States
(12) Patent Application Publication
(10) Pub. No.: US 2024/0001371 A1
(43) Pub. Date: Jan. 4, 2024

(54) METHOD FOR ENSURING MICROBIOLOGICAL PURITY OF A SINGLE-USE DEVICE, AND COVER FOR A FLUID CONNECTION OF A SEPARATION UNIT FOR USE IN SUCH A METHOD

(71) Applicant: Sartorius Stedim Biotech GmbH, Goettingen (DE)

(72) Inventors: Johannes WORTMEYER, Goettingen (DE); Thomas LOEWE, Goettingen (DE); Florian MIETH, Goettingen (DE)

(21) Appl. No.: 18/037,154

(22) PCT Filed: Nov. 16, 2021
(86) PCT No.: PCT/EP2021/081835

§ 371 Date: May 16, 2023

(30) Foreign Application Priority Data
Nov. 16, 2020 (EP) 20207785.5

(51) Int. Cl. B01L 3/00; A61L 2/08; A61L 2/07

(52) U.S. Cl. CPC B01L 13/02; B01L 3/565; B01L 2200/0689; A61L 2/081; A61L 2/07; B01L 3/561

(57) ABSTRACT

A method for ensuring a microbiological purity of a single-use device for carrying out a biotechnological process, the single-use device including at least one gamma-sterilizable component which is formed from materials suitable for a sterilization by gamma radiation, and at least one non-gamma-sterilizable subunit which contains a material unsuitable for a sterilization by gamma radiation. Both the component and the subunit each have an area that comes into contact with a process medium when the biotechnological process is carried out. The method includes the steps of sterilizing the gamma-sterilizable component by gamma radiation; protecting the medium-contacting area of the gamma-sterilizable component by a sterile barrier; sterilizing the non-gamma-sterilizable subunit with superheated steam; protecting the medium-contacting area of the non-gamma-sterilizable subunit by a sterile barrier; removing the sterile barriers; and mounting the gamma-sterilized component and the superheated steam-sterilized subunit in the single-use device immediately after removing the sterile barriers.

### FIELD OF THE INVENTION

The invention relates to a method for ensuring a microbiological purity of a single-use device for carrying out a biotechnological process, in particular a single-use filtration device. The invention further relates to a lid for a fluid connection of a separation unit. Furthermore, the invention relates to a use of a lid for a fluid connection of a separation unit in a method for ensuring a microbiological purity of a single-use device.

### BACKGROUND

The present invention is directed to complex devices of large dimensions for commercial manufacturing (production scale). Such devices are to be delimited in particular from smaller devices which are easier to handle and are used for experimental purposes or for developing new processes (laboratory scale).

A single-use device within the meaning of the invention is a device intended for one-time use which is not subsequently cleaned and sterilized for reuse, but is usually disposed of completely. Accordingly, a single-use device differs in terms of the materials (mainly plastic materials) used for its individual components from a reusable device which includes, e.g., components made of stainless steel.

In the context of the present invention, a separation unit is understood to mean, e.g., a filter capsule, a chromatography column or a membrane adsorber. In principle, however, the invention is also applicable in single-use devices with other functional units, in particular if—in contrast to other components of the single-use device—these units are not suitable for a sterilization by gamma radiation. This will be discussed later.

Basically, a user of a single-use device for carrying out a biotechnological process, in particular a single-use filtration device, wishes the single-use device to be ready for use immediately after delivery. A prerequisite for this is usually that the single-use device meets specific requirements for microbiological purity. In many applications, this is indispensable, because otherwise there is the risk that the operability or capability of the separation units is not ensured and/or that the target product is contaminated. The latter is particularly critical if the contamination is similar to the target product and thus cannot be effectively filtered out subsequently.

It involves an additional burden to the user, however, if he or she has to ensure the desired microbiological purity of the single-use device himself or herself. It is therefore desirable for the single-use device to be sterilized already on the part of the manufacturer. In order for the single-use device to be delivered to the user in a sterile condition, it must be provided with a sterile barrier, which can be used as a packaging for transport at the same time.

However, sterilization of an entire single-use device can be problematic for a variety of reasons. If a single-use device contains separation units or other functional units that cannot be sterilized in the same way as the other medium-contacting components of the single-use device, separate sterilization processes are required for the respective components. This means that the single-use device cannot be sterilized as a whole after assembly.

In principle, sterilization by gamma radiation is advantageous since it does not affect the dimensions of the components (dimensional stability). However, certain materials that are used in separation units, e.g. as a filter material or membrane material, such as polytetrafluoroethylene (PTFE), polypropylene (PP) or polyvinyl chloride (PVC), are not suitable for sterilization by gamma radiation since they lose important material properties as a result of the irradiation, in particular their mechanical stability. This is of importance, e.g., for components that are subject to heavy or abrupt mechanical stress, such as connectors on hose ends that may fall to the floor. Likewise, in the case of fluid-carrying components, critical contamination by “extractables” (chemical compounds that are extracted from the material of the component under extreme conditions) must not occur as a result of gamma irradiation. Also not suitable for gamma sterilization are optical components, e.g. sight glasses or ports made from transparent or translucent plastic materials for optical sensors or for visual inspection, which become discolored when irradiated.

Separation units (and other components) that do not come into consideration for gamma sterilization due to their materials or for the further reasons mentioned above therefore have to be sterilized by other means, preferably by superheated steam in an autoclave. However, superheated steam sterilization has the drawback that the overpressure in the autoclave can cause undesirable plastic deformation of the separation units. Plastic clamps and clips, for example, which produce a press-fit connection, are generally not suitable for autoclaving. The plastic materials creep strongly at high temperatures, so that the tightness of the press-fit connection can no longer be ensured. Retightening of the clamp or clip is usually only possible to a limited extent or not at all.

Non-heat-resistant materials such as polyethylene (PE), from which connectors or containers are produced, for example, are not autoclavable but gamma-sterilizable. Also not autoclavable are bolted or bonded components if the thermal expansions differ greatly. Pipes or hoses made from thermoplastic elastomers, for example, can only be autoclaved to a limited extent, since after autoclaving they remain “frozen” in the bent state in which they had been placed in the autoclave.

In any case, a person skilled in the art can assess whether or not a component intended for a biotechnological process is suitable for gamma sterilization or autoclaving, based on the materials from which the component is formed and based on the intended use of the component.

If the single-use device is to be delivered as a whole, there is furthermore the problem that before, during and after the assembly of the separation units sterilized in separate processes and the other components, contaminants may reach the interior of the separation units and of the other components.

Against this background, it is the object of the invention to improve and simplify ensuring a microbiological purity of a single-use device for carrying out a biotechnological process, in particular a single-use filtration device, before it is put into operation.

### SUMMARY

This object is achieved by a method having the features of claim 1 and by a method having the features of claim 3. Advantageous and expedient configurations of the method according to the invention are given in the appertaining dependent claims.

The method according to the invention for ensuring a microbiological purity is intended for a single-use device for carrying out a biotechnological process, the single-use device including at least one gamma-sterilizable component which is formed from materials suitable for a sterilization by gamma radiation, and at least one non-gamma-sterilizable subunit which contains a material unsuitable for a sterilization by gamma radiation. Both the component and the subunit each have an area that comes into contact with a process medium when the biotechnological process is carried out. According to a first aspect, the method according to the invention includes the steps of: (a) sterilizing the gamma-sterilizable component by gamma radiation; (b) protecting the medium-contacting area of the gamma-sterilizable component by a sterile barrier; (c) sterilizing the non-gamma-sterilizable subunit with superheated steam; (d) protecting the medium-contacting area of the non-gamma-sterilizable subunit by a sterile barrier; (e) removing the sterile barriers; and (f) mounting the gamma-sterilized component and the superheated steam-sterilized subunit in the single-use device immediately after removing the sterile barriers.

The invention is based on the finding that in a single-use device, in particular a single-use device having one or more separation units, which cannot be sterilized as a whole after assembly, the risk of contamination is highest in the phase before and during assembly of the single-use device. The invention therefore provides for special protective measures:

The gamma-sterilizable component and the non-gamma-sterilizable subunit are sterilized in separate processes, and the medium-contacting areas of the gamma-sterilizable component and of the non-gamma-sterilizable subunit are each protected by a sterile barrier. Such a sterile barrier can be, in particular, an envelope or a packaging for the entire component or subunit, or a closure, such as a blind cap, a sterile connector or a jacket, which protects the respective medium-contacting area, as will be discussed in more detail later.

The sterile barriers will not be removed until immediately prior to the mounting of the gamma-sterilized component and the superheated steam-sterilized subunit.

Provided that no further protective measures are provided after the removal of the sterile barriers, such as additional closures, which will be discussed later, the time period between removing a sterile barrier and mounting the associated gamma-sterilized component or the associated superheated steam-sterilized subunit should be shorter than four minutes, preferably shorter than two minutes, further preferably shorter than one minute, and still further preferably shorter than half a minute, depending on the particle concentration in the environment. Practical tests have shown that when these time periods are observed, the risk of contamination of the medium-contacting areas of the single-use device is very effectively reduced.

Basically, the at least one non-gamma-sterilizable subunit and the at least one gamma-sterilizable component may be sterilized at different times, without an order having to be observed.

According to a second aspect of the invention, in addition to the sterile barriers (in some circumstances, a shared sterile barrier may be provided for both the at least one gamma-sterilizable component and the at least one non-gamma-sterilizable subunit), at least one closure is employed which covers the medium-contacting area of the gamma-sterilizable component or of the non-gamma-sterilizable component. Such a closure, which is independent of the sterile barrier, need not constitute a sterile barrier as long as it at least contributes to significantly reducing the likelihood of undesirable particle input. After removal of the sterile barrier, owing to the additional closure an extended time window then remains for mounting the component or subunit concerned.

According to this second aspect, the method according to the invention includes the steps of: (a) sterilizing the gamma-sterilizable component by gamma radiation; (b) protecting the medium-contacting area of the gamma-sterilizable component by a sterile barrier; (c) sterilizing the non-gamma-sterilizable subunit with superheated steam; (d) protecting the medium-contacting area of the non-gamma-sterilizable subunit by a sterile barrier; (e) additionally covering the medium-contacting area of the gamma-sterilizable component with at least one first closure; and/or additionally covering the medium-contacting area of the non-gamma-sterilizable subunit with at least one second closure; (f) removing the sterile barriers and later removing the first and/or second closure; and (g) mounting the gamma-sterilized component and/or the superheated steam-sterilized subunit in the single-use device immediately after removing the first and second closures, respectively.

As used herein, the designations “first” and “second” closures merely serve to distinguish a closure provided for the gamma-sterilizable component from a closure provided for the non-gamma-sterilizable subunit. That is, a “first” closure does not absolutely require a “second” closure, and vice versa.

Specifically, the extended time period between removing the respective sterile barrier and mounting the associated gamma-sterilized component provided with a first closure and/or the associated superheated steam-sterilized subunit provided with a second closure may in each case be shorter than three hours, preferably shorter than two hours, further preferably shorter than one hour and further preferably shorter than half an hour.

The time period between removing the first and second closures, respectively, and mounting the associated gamma-sterilized component and superheated steam-sterilized subunit, respectively, should, however, in each case be again shorter than two minutes, preferably shorter than one minute, and further preferably shorter than half a minute.

If, in addition to the sterile barrier, at least one closure is additionally provided to cover the medium-contacting area of the gamma-sterilizable component or of the non-gamma-sterilizable component, such a closure should, for effective protection, constitute a particle input reducing barrier having a clearance of at most 100 μm, preferably of at most 50 μm and further preferably of at most 10 μm. In principle, it is also possible to use a closure which itself constitutes a sterile barrier.

In the method according to the invention, it may furthermore be provided that step (b) is performed before step (a) and/or step (d) is performed before step (c), i.e. the medium-contacting areas of the gamma-sterilizable component or of the non-gamma-sterilizable subunit can be protected with a sterile barrier even prior to the sterilizing process. Accordingly, for the gamma-sterilizable component, a gamma radiation-resistant material has to be selected for the sterile barrier. In the case of the subunit sterilized with superheated steam, it has to be ensured that the superheated steam can penetrate the sterile barrier to a sufficient degree. In both cases, it is then ensured that after the sterilized component or subunit has been transferred to a non-sterile environment, the medium-contacting areas will each remain sterile.

Generally, a closed envelope completely surrounding the subunit and formed from a nonwoven fabric, preferably Tyvek®, is suitable for use as a sterile barrier for the subunit that is sterilized with superheated steam. This nonwoven fabric consists of high-density polyethylene (PE-HD) and is composed of fibrillated ultrafine filaments in the diameter range of from 0.5 to 10 μm that are tightly connected with one another to form crosslinks. Such a material distinguishes itself by its high water vapor permeability and thus allows rapid penetration of superheated steam, which is required for the sterilization of the subunit located in the closed envelope. On the other hand, the material is suitable as a sterile barrier under normal ambient conditions. The material loses only very few fibers, and it is hydrophobic, i.e. water does not permeate the material. For this reason, the material retains its strength, irrespective of whether it is wetted with water or dry. The subunit can therefore be sterilized with superheated steam within an envelope formed from such a material and can subsequently be stored and/or transported.

According to a particular aspect of the invention, the medium-contacting area of the subunit is covered by a second closure, in particular a lid, before sterilization with superheated steam and until the superheated steam-sterilized subunit is mounted. The closure has at least one passage opening which can be selectively uncovered and closed. As already mentioned at the outset, this closure can form the sterile barrier, provided in accordance with the invention, for the medium-contacting part of the subunit. However, this is not absolutely necessary if the sterile barrier is formed by other means, in particular by the aforementioned envelope.

With a view to the preferred application of the invention, the non-gamma-sterilizable subunit includes at least one functional unit, in particular a separation unit (e.g., a filter capsule, a chromatography column or a membrane adsorber), having at least one fluid connection. When the method according to the invention is carried out, the fluid connection is preferably covered by a second closure. If additionally a sterile barrier is provided, such as the aforementioned envelope, the exposure of the fluid connection can be shortened after removing the envelope, by removing the closure only immediately prior to mounting the functional unit. This means that the time interval available for a probably “clean” (uncontaminated) installation is extended, since after removing the sterile barrier, it is not necessary to assemble all fluid connections within two minutes. This is of advantage in particular in the case of complicated setups involving many connections, since one closure after the other can be removed in the course of installation.

In a particularly preferred embodiment, the method according to the invention includes the following further steps or measures: attaching the second closure onto the fluid connection of the functional unit; uncovering the passage opening if the passage opening is closed; packing the non-gamma-sterilizable subunit into the envelope; sterilizing the packed subunit with superheated steam with the passage opening uncovered; after sterilizing with superheated steam: closing the passage opening in the closed envelope; unpacking the superheated steam-sterilized subunit from the closed envelope; and removing the second closure immediately prior to mounting the subunit.

This means that the subunit along with the functional unit, in particular a separation unit, is first packaged in an envelope and then sterilized in the closed envelope so that no contamination can enter the envelope after the sterilizing process. This is possible because, as discussed earlier, the envelope can be produced from a material that is permeable to superheated steam but acts as a sterile barrier under normal conditions.

A further prerequisite is that the functional unit does not deform plastically as a result of the superheated steam sterilization. This is achieved in that the passage opening of the second closure applied prior to sterilization is exposed during the superheated steam sterilization, so that a pressure equalization is possible through the open fluid connection of the separation unit.

In addition, it is ensured that after unpacking the subunit from the envelope, a particle input reducing barrier continues to exist, which protects the medium-contacting area from contamination. This barrier is provided by the second closure, the passage opening of which was closed after sterilization in the closed envelope. Therefore, after the superheated steam-sterilized subunit has been unpacked, protection is still provided until the second closure is removed in the course of assembling the single-use device.

As already discussed, according to the invention, the at least one gamma-sterilizable component of the single-use device is sterilized separately, i.e. not together with the non-gamma-sterilizable subunit, since sterilization with superheated steam involves the risk that thereafter the dimensional stability of the component is no longer given. However, dimensional stability is of essential importance, especially for a component that is critical in this respect, such as a rigid connecting pipe having fluid connections to which a plurality of separation units fixed in a rigid holder and further connecting pipes are to be connected. The dimensional stability is not impaired in the case of sterilization of the component by gamma radiation as provided according to the invention.

In order to minimize the risk of contamination of the medium-contacting area of the gamma-sterilizable component of the single-use device even after removal of the sterile barrier provided according to the invention, provision is made in accordance with the second aspect of the invention for the medium-contacting area of the component to remain covered by a first closure, preferably by a blind cap, a sterile connector or a jacket. Since the gamma radiation does not—neither in the short nor in the long term—affect the dimensional stability of the component and the closure, provided the latter is formed from a suitable material, (no elastic or plastic deformation, no brittleness or the like), the closure may already be applied prior to the sterilizing process and remain there until the component is mounted. As a result, protection is provided for the medium-contacting area of the gamma-sterilized component until the first closure is removed in the course of mounting the component. However, in accordance with the first aspect of the invention, the first closure for covering the medium-contacting area of the gamma-sterilizable component may also constitute the sole sterile barrier for the component.

With regard to the preferred application of the invention, the at least one gamma-sterilizable component typically comprises a connecting pipe and/or a plurality of interconnectable manifold assemblies for connecting a plurality of separation units and/or at least one connecting hose line, wherein the gamma-sterilizable component includes at least one fluid connection that is covered by the first closure as described above.

If no first closure is provided for the gamma-sterilizable component or such a closure does not form a sterile barrier, the sterile barrier provided according to the invention for the medium-contacting part of the component is preferably formed by a closed primary packaging completely surrounding the component.

To further minimize the risk of contamination from the environment, the superheated steam-sterilized subunit and the gamma-sterilized component should be mounted on a biological safety cabinet (laminar flow cabinet, clean bench) or in a clean room, preferably in an area of a clean room separated by walls.

The invention also provides a lid for a fluid connection of a non-gamma-sterilizable subunit for use in a method according to the invention, wherein a second closure is provided for a separation unit of the subunit. The lid according to the invention comprises a base body for accurately fitting the lid to the fluid connection. The lid has at least one passage opening that provides a fluid communication between the medium-contacting area of the separation unit and the immediate surroundings of the separation unit after the lid has been attached to the fluid connection. The lid further comprises a mechanism for selectively uncovering and closing the at least one passage opening.

A lid of this type can be attached to the fluid connection of the separation unit prior to the superheated steam sterilization and remain there until the single-use device is assembled. The special closure mechanism allows the fluid connection to be selectively brought into fluid communication with the environment or separated from the environment (at least to counteract any undesirable particle input) without the lid having to be removed from the fluid connection for the uncovering and closing processes. As already discussed above, in the method according to the invention, it is provided that during sterilization with superheated steam, the passage opening is in an uncovered state to allow pressure equalization. Thereafter, the passage opening is closed in order to form the desired particle barrier against the environment.

According to a preferred design of the lid according to the invention, the mechanism includes a closing body which is transferable between a closed position, in which the closing body covers the at least one passage opening, and an open position, in which the closing body does not cover the at least one passage opening. This means that covering and uncovering is not effected by attaching and removing the lid, but in each case by a movement of the closing body with the lid attached.

The mechanism for covering and uncovering the passage opening should be manually actuatable even when the separation unit is in the closed envelope. To this end, the lid has an actuating element that is adapted to be easily felt and grasped.

In contrast to the separation unit itself, the lid may be a reusable component which is preferably at least mainly formed of stainless steel. If the lid is no longer needed after the single-use device has been set up, it can be employed for the same purpose in another single-use device after appropriate cleaning.

### BRIEF DESCRIPTION OF THE DRAWINGS

Further features and advantages of the invention will be apparent from the description below and from the accompanying drawings, to which reference is made and in which:

FIG. 1 shows a perspective view of a single-use device for carrying out a biotechnological process;

FIG. 2 shows a top view of the single-use device from FIG. 1;

FIG. 3 shows a separation unit with connecting hose lines and connectors;

FIG. 4 shows a lid for a fluid connection of a separation unit;

FIG. 5 shows an exploded view from the side of a connecting pipe with a plurality of fluid connections; and

FIG. 6 shows a top view of the connecting pipe of FIG. 5.

### DETAILED DESCRIPTION

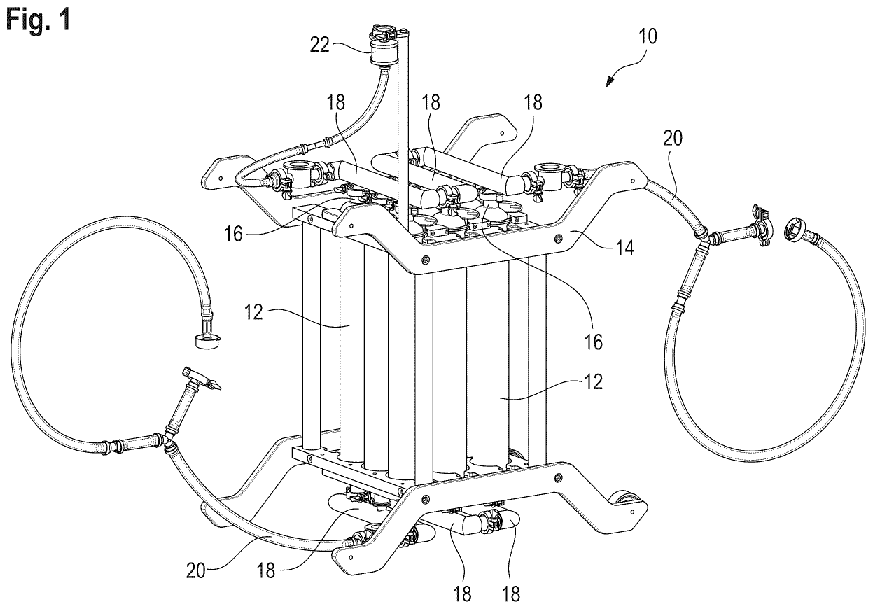

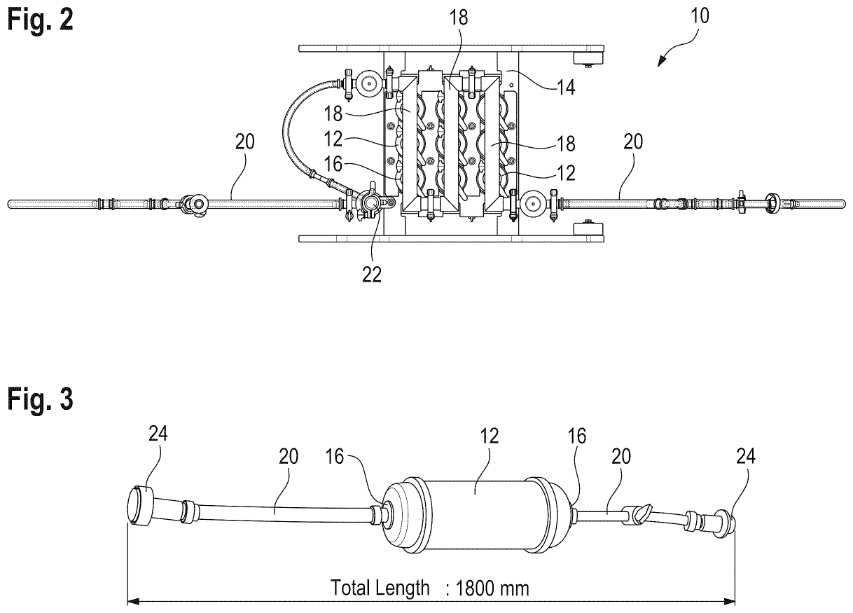

FIGS. 1 and 2 show a single-use device 10 for carrying out a biotechnological process in an assembled state.

The single-use device 10 comprises a plurality of separation units 12 arranged in a rigid holder 14 in a predefined grid. The separation units 12 may generally be filter capsules, chromatography columns, or membrane adsorbers.

The fluid connections (inlets and outlets) 16 of the separation units 12 are interconnected via rigid connecting pipes 18 or hose lines. Connecting hose lines 20 are connected to the connecting pipes 18 for supplying and discharging medium.

Furthermore, a sterile air filter 22 is connected to an upper connecting pipe 18 for jointly venting all of the separation units 12.

The single-use device 10 may include still further components. However, the exact structure and operation of the single-use device 10 are not of essential importance to the invention described herein.

The single-use device 10 is a “large” device for use in commercial manufacturing involving a large media throughput (production scale). In such devices, a plurality of separation units are connected to each other, in particular more than three 30-inch filter capsules (approx. 850 mm in length). This is to be distinguished from smaller devices that are used in laboratories for experimental purposes or process development (laboratory scale).

The single-use device 10 is intended to be delivered to the user as a whole and, as far as possible, in a sterile condition or at least with as low a germ load (bioburden) and other contamination by particles or the like as possible. Basically, in this connection, a sterilization by gamma radiation is preferred, since with this method the dimensional accuracy of the components can, as a rule, be guaranteed.

However, in the present case, the single-use device 10 includes at least one separation unit 12 that is not suitable for a sterilization by gamma radiation. This is the case, in particular, if the filter material or membrane material would be so severely damaged by the energy dose (e.g., 25 kGy) typically administered during gamma sterilization that its intended function can no longer be guaranteed. Examples of such unsuitable materials are polytetrafluoroethylene (PTFE), polypropylene (PP) and polyvinyl chloride (PVC).

The separation units 12 not sterilizable by gamma radiation are sterilized as subunits in an autoclave using superheated steam, either individually or in combination and, if desired, together with further components. Such a subunit may only be of a size that allows it to be accommodated in the autoclave.

When dividing the single-use device 10 into subunits, a further criterion has to be taken into account in addition to the external dimensions: It must be ensured that, during the treatment in the autoclave, especially the inner surfaces of the separation units 12 come into contact with the superheated steam to a sufficient extent and for a long enough time. This is of particular importance if a plurality of separation units 12 are connected to one another, such as by the connecting pipes 18, or are connected to hose lines 20, so that the superheated steam cannot enter the separation units 12 directly through the fluid connections 16. There is then basically the risk that the superheated steam does not, or not quickly enough, reach all of the relevant surfaces to ensure sufficient sterilization.

In practice, it has been found that the total length of a subunit through which the superheated steam has to flow should not exceed 1800 mm. This is shown in FIG. 3 by reference to an example in which the total length through which flow has to pass, of a filter capsule 12 with two connected hose lines 20 and connectors 24 at the free ends, is approximately 1800 mm.

In the following, it will be described on the basis of a preferred exemplary embodiment how a prespecified purity, in particular a prespecified microbiological purity, of the single-use device 10 is ensured until the entire single-use device 10 is put into operation on a user's premises.

First, the superheated steam sterilization of a subunit having a separation unit 12 is described. If applicable, this description is to be transferred to the further separation units 12 of the same subunit or to the separation units 12 of further subunits.

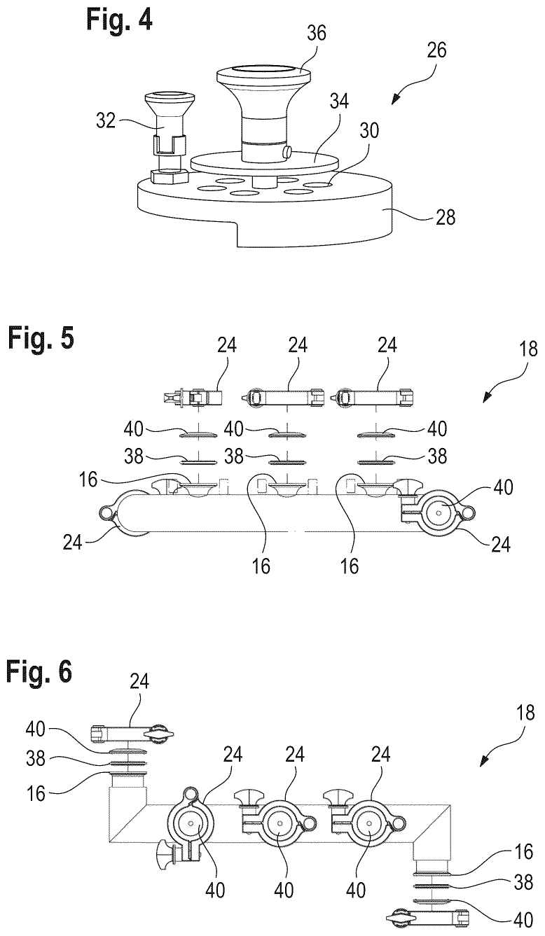

Those face sides of the separation unit 12 at which one or more free fluid connections 16 are located (i.e. fluid connections 16 to which no connecting pipe 18 and no hose line 20 or the like is connected prior to sterilization) are provided with a closure. In the exemplary embodiment described herein, a lid 26 as shown in FIG. 4 is employed as the closure. The lid 26, which is formed primarily of stainless steel, is a reusable component, i.e. it can be used for further sterilization processes.

The lid 26 essentially consists of a base body 28 having one or more through passage openings 30, a locking pin 32, and a closure mechanism for selectively exposing and closing the passage openings 30.

The base body 28 is precisely matched to the outer contour of the associated face side of the separation unit 12 and can be fixed in place at the separation unit 12 by means of the locking pin 32.

The closure mechanism includes a closing body 34 which can assume two defined, stable positions: a closed position, in which the closing body 34 firmly rests against the base body 28 and covers the passage openings 30, and an open position, in which the closing body 34 is raised so that it does not cover the passage openings 30.

The closure mechanism is adapted to be manually actuated by means of an actuating element 36, i.e. the closing body 34 can be transferred to the respective other position by pushing or pulling the actuating element 36.

In the fixed position of the lid 26, the fluid connection(s) 16 of the separation unit 12 are in fluid communication with the immediate environment of the separation unit 12 via the passage openings 30 when the closing body 34 is in the open position. When the closing body 34 is in the closed position, however, there is no, or virtually no, fluid communication between the interior of the separation unit 12 and the environment, i.e. a possible particle input is largely prevented. The base body 28 and the closing body 34 are designed such that in the closed position the lid 26 has a clearance of at most 100 μm, preferably at most 50 μm and further preferably at most 10 μm, so that correspondingly larger particles are prevented from entering.

After attaching the lid 26 onto the face side of the separation unit 12 (and, if applicable, another lid 26 onto the other face side), the subunit is packed into an envelope, ensuring beforehand that the passage openings 30 are exposed. The envelope is made of Tyvek® or a comparable material which, on the one hand, is watertight and can serve as a sterile barrier under normal ambient conditions but, on the other hand, is permeable to water vapor. The envelope is closed by welding.

Subsequently, the subunit inside the envelope is placed in the autoclave and sterilized with superheated steam under predefined conditions (e.g., 40 min at 121° C. or 30 min at 131° C.). The superheated steam passes through the envelope and reaches the outer surfaces of the subunit and through the uncovered passage openings 30 into the interior of the separation unit 12. The passage openings 30 are dimensioned to be sufficiently large for this purpose. The passage openings 30 also ensure that sufficient pressure equalization can take place during the superheated steam sterilization so that the separation unit 12 does not deform plastically.

The envelope will remain closed after sterilization and serves as a sterile barrier for the subunit until the single-use device 10 is assembled.

The remaining components of the single-use device 10 are sterilized by gamma radiation before being assembled. Prior to irradiation, the open fluid connections 16 of the connecting pipes 18 and of the connecting hose lines 20 are also covered by closures, in this case in the form of seals 38 and blind caps 40, which are fitted by means of connectors 24, such as tri-clamp connectors. This is shown in FIGS. 5 and 6 using a Z-shaped connecting pipe 18 as an example. The closures and any aids for fastening them are made of a gamma radiation-resistant material and are designed so as to effectively prevent any particle ingress through the fluid connections 16, with the clearance dimensions being comparable to the preferred clearance dimensions for the fluid connection 16, covered by the lid 26, of the separation unit 12. In principle, the closures may also be designed as sterile barriers.

The connecting pipes 18 and connecting hose lines 20 are then packed in a primary packaging which serves as a sterile barrier. Sterilization of these components is then performed in the packaged state, which is why for the primary packaging a material should also be selected the properties of which are not significantly impaired by gamma radiation in the dose customary for a sterilization.

The single-use device 10 is assembled either at the manufacturer's or—after transport of the sterile-packaged subunits and further components—at the user's, and preferably on a biological safety cabinet that allows sterile connecting, or in an area of a clean room separated by walls.

Prior to unpacking the subunit from the envelope, the passage openings 30 of the lid 26 are closed. This is done with the envelope closed by grasping the actuating element 36 and actuating the closure mechanism. Despite the envelope being located in between, the distinctive actuating element 36 can be easily located by feeling it.

By closing the passage openings 30, the lid 26 provides protection against particle input and, if required, even a sterile barrier, even after the subunit has been unpacked, so that an ingress of contaminants into the interior of the separation unit 12 is largely prevented.

Likewise, after unpacking, the blind caps 40 serve as a protection against particle input, preferably even as sterile barriers, and in this way largely prevent any contaminants from being able to penetrate into the interior of the connecting pipes 18 or the connecting hose lines 20.

Nonetheless, when assembling the single-use device 10, the envelope or the primary packaging should in each case be opened as late as possible in order to keep the exposure to the environment of the fluid connections 16 covered by the closures as short as possible. Owing to the closures, however, the exposure may be up to a total of three hours. However, less than two hours is preferred, even better less than one hour and ideally less than half an hour.

During this time, each separation unit 12 is placed in its intended position in the rigid holder 14. The connecting pipes 18 and connecting hose lines 20, if not already preassembled, are laid out ready. Only immediately before connecting a component are the lids 26 and/or blind caps 40 removed from the fluid connections 16 required for the respective connection. After removal of a lid 26 or a blind cap 40, the exposure of the respective open fluid connection 16 to the environment should be kept significantly shorter. Preferably, this time interval is less than two minutes, better, less than one minute and, ideally, less than 30 seconds. Assembly in a cleanroom class better than ISO 8 is then not imperative under these preconditions.

Usually, first the connecting pipes 18 are connected to the separation units 12 in the manner described above, before the connecting hose lines 20 are then connected to the connecting pipes 18. In any case, a self-contained system has been established in the end.

If the assembly is already carried out at the manufacturer's, that is, not at the later user's, the assembled single-use device 10 is packaged as a whole in a package serving as a sterile barrier and delivered to the user.

After the single-use device 10 has been set up and/or assembled on the user's premises, the blind caps 40 on the free hose ends remain in place until the connecting hose lines 20 are connected to a medium supply and/or discharge as part of putting the device into operation.

Further safety measures for the work steps described above during the assembly of the single-use device 10 require the wearing of sterile gloves and a protective mask. The otherwise usual cleanroom clothing is obligatory.

Two persons are intended for the assembly of the single-use device 10, in particular in order to achieve the desired short exposure of open fluid connections 16. The first person is in charge of unpacking and making the components ready. The second person removes the lids 26 and the blind caps 40 from the fluid connections 16 and establishes the intended fluid communications, i.e., works primarily at those locations that are critical to the cleanliness inside the components through which medium flows.

In addition, work surfaces, tools and storage containers are cleaned separately.

The single-use device 10 with its separation units and other components has been described by way of example only. In addition or as an alternative to the separation units 12, other functional units may also be provided that are not suitable for sterilization by gamma radiation, such as sensor arrangements having electronic data storage devices which would be impaired by gamma radiation.

In place of the lid 26 described, a different closure having a comparable functionality may also be used for the separation units 16 or other functional units. An example of this is a plug made of silicone or a similar material with one or more slits, which is fitted on a fluid connection 16 of a separation unit 12. During heating in the autoclave, the plug will expand and the slits will form passage openings so that superheated steam can enter the interior of the separation unit 12 through the passage openings formed in this way. During subsequent cooling, the plug will shrink back to its normal size so that the passage openings will close and an ingress of contaminants through the slits is largely avoided. It is also possible to place an air-permeable or superheated steam-permeable dust bag around the fluid connection 16 as a protection and to fix it in place there.

For (selectively) connecting the separation units 12 and/or functional units, a plurality of directly interconnectable, gamma-sterilizable manifold assemblies may also be used, which are attached directly to the face sides of the separation units 12 or functional units.

### LIST OF REFERENCE NUMBERS

10 single-use device
12 separation unit
14 holder
16 fluid connection
18 connecting pipe
20 (connecting) hose line
22 sterile air filter
24 connector
26 lid
28 base body
30 passage opening
32 locking pin
34 closing body
36 actuating element
38 seal
40 blind cap

1. A method of ensuring a microbiological purity of a single-use device for carrying out a biotechnological process, wherein the single-use device comprises at least one gamma-sterilizable component which is formed from materials suitable for a sterilization by gamma radiation, and at least one non-gamma-sterilizable subunit which contains a material unsuitable for a sterilization by gamma radiation, both the component and the subunit each having a medium-contacting area that comes into contact with a process medium when the biotechnological process is carried out, and wherein the method comprises steps of:
(a) sterilizing the gamma-sterilizable component by gamma radiation;
(b) protecting the medium-contacting area of the gamma-sterilizable component by a sterile barrier;
(c) sterilizing the non-gamma-sterilizable subunit with superheated steam;
(d) protecting the medium-contacting area of the non-gamma-sterilizable subunit by a sterile barrier;
(e) removing the sterile barriers; and
(f) mounting the gamma-sterilized component and the superheated steam-sterilized subunit in the single-use device immediately after removing the sterile barriers.

2. The method according to claim 1, characterized in that a time period between removing a sterile barrier and mounting the associated gamma-sterilized component or the associated superheated steam-sterilized subunit, respectively, is in each case shorter than four minutes.

3. A method of ensuring a microbiological purity of a single-use device for carrying out a biotechnological process, wherein the single-use device comprises at least one gamma-sterilizable component which is formed from materials suitable for a sterilization by gamma radiation, and at least one non-gamma-sterilizable subunit which contains a material unsuitable for a sterilization by gamma radiation, both the component and the subunit each having a medium-contacting area that comes into contact with a process medium when the biotechnological process is carried out, and wherein the method comprises steps of:
(a) sterilizing the gamma-sterilizable component by gamma radiation;
(b) protecting the medium-contacting area of the gamma-sterilizable component by a sterile barrier;
(c) sterilizing the non-gamma-sterilizable subunit with superheated steam;
(d) protecting the medium-contacting area of the non-gamma-sterilizable subunit by a sterile barrier;
(e) additionally covering the medium-contacting area of the gamma-sterilizable component with at least one first closure, and/or additionally covering the medium-contacting area of the non-gamma-sterilizable subunit with at least one second closure;
(f) removing the sterile barriers and later removing the first and/or second closure; and
(g) mounting the gamma-sterilized component and/or the superheated steam-sterilized subunit in the single-use device immediately after removing the first and second closures, respectively.

4. The method according to claim 3, characterized in that
a time period between removing the respective sterile barrier and mounting the associated gamma-sterilized component provided with a first closure and/or the associated superheated steam-sterilized subunit provided with a second closure is in each case shorter than three hours, and
in that the time period between removing the first and second closures, respectively, and mounting the associated gamma-sterilized component and superheated steam-sterilized subunit, respectively, is in each case shorter than two minutes.

5. The method according to claim 3, characterized in that at least one of the first and second closures constitutes a particle input reducing barrier having a clearance of at most 100 μm.

6. The method according to claim 1, characterized in that step (b) is performed before step (a) and/or step (d) is performed before step (c).

7. The method according to claim 1, characterized in that the sterile barrier for the superheated steam-sterilized subunit comprises a closed envelope completely surrounding the subunit.

8. The method according to claim 7, characterized in that the envelope is formed from a watertight but water vapor-permeable nonwoven fabric.

9. The method according to claim 1, characterized in that the medium-contacting area of the subunit is covered by a second closure, the second closure being a lid, before sterilization with superheated steam and until the superheated steam-sterilized subunit is mounted, the second closure having at least one passage opening which can be selectively uncovered and closed.

10. The method according to claim 9, characterized in that the non-gamma-sterilizable subunit includes at least one functional unit, the at least one functional unit being a separation unit, having a fluid connection which is covered by a second closure.

11. The method according to claim 10, characterized by the following steps or measures:
attaching the second closure onto the fluid connection of the functional unit;
uncovering the passage opening if the passage opening is closed;
packing the non-gamma-sterilizable subunit into a closed envelope;
sterilizing the packed subunit with superheated steam with the passage opening uncovered;
after sterilizing with superheated steam: closing the passage opening in the closed envelope;
unpacking the superheated steam-sterilized subunit from the closed envelope; and
removing the second closure immediately prior to mounting the subunit.

12. The method according to claim 1, characterized in that the medium-contacting area of the gamma-sterilizable component remains covered by a first closure, by a blind cap, a sterile connector or a jacket, prior to the sterilization by gamma radiation and until the gamma-sterilized component is mounted.

13. The method according to claim 12, characterized in that the gamma-sterilizable component comprises at least one connecting pipe and/or a plurality of interconnectable manifold assemblies for connecting a plurality of separation units and/or at least one connecting hose line, wherein the gamma-sterilizable component includes at least one fluid connection that is covered by the first closure.

14. The method according to claim 1, characterized in that the sterile barrier for the gamma-sterilizable component comprises a closed primary packaging completely surrounding the component.

15. The method according to claim 1, characterized in that the superheated steam-sterilized subunit and the gamma-sterilized component are mounted on a biological safety cabinet or in a clean room, in an area of a clean room that is separated by walls.

16. A lid for a fluid connection of a separation unit of a non-gamma-sterilizable subunit for use in a method according to claim 3, the lid comprising:
a base body for accurately fitting the lid to the fluid connection;
at least one passage opening that provides a fluid communication between the medium-contacting area of the separation unit and immediate surroundings of the separation unit after the lid has been attached to the fluid connection; and
a mechanism for selectively uncovering and closing the at least one passage opening, the mechanism including a closing body which is transferable between a closed position, in which the closing body covers the at least one passage opening, and an open position, in which the closing body does not cover the at least one passage opening; and
an actuating element which is used for manually actuating the mechanism;
wherein the lid is a reusable component, which is further formed at least mainly of stainless steel.

17. (canceled)
